(19) United States
(12) Patent Application Publication
(10) Pub. No.: US 2010/0152771 A1
(43) Pub. Date: Jun. 17, 2010

(54) Disposable acupressure relief strip

(76) Inventor: Joseph Di Lustro, Tinton Falls, NJ (US)

Correspondence Address: Charles I. Brodsky, 2 Buck Lane, Marlboro, NJ, 07746, US

(21) Appl. No.: 12/658,717

(22) Filed: Feb. 16, 2010

Related U.S. Application Data
(63) Continuation-in-part of application No. 11/490,274, filed on Jul. 21, 2006.

(51) Int. Cl. A61B 17/00

(52) U.S. Cl. 606/204

(57) ABSTRACT

A disposable acupressure strip for nausea relief in humans, dogs & cats includes a raised portion of a contact button extending from a film substrate through a cutout in a paper liner positioned over an acupressure point on a user's wrist.

### CROSS-REFERENCE TO RELATED APPLICATIONS

This application is a Continuation-in-Part of application Ser. No. 11/490,274 filed Jul. 21, 2006.

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

NOT APPLICABLE

### BACKGROUND OF THE INVENTION

1. Field of the Invention

This invention relates to acupressure sensory relief, in general, and to a disposable acupressure strip for use with people, dogs and cats, in particular.

2. Description of the Related Art

As is understood in the medical and surgical community, approximately 50 percent of all surgical procedures are impacted by postoperative nausea and vomiting (PONV). The usual procedure to deal with this is for an anesthesiologist to provide an anti-sickness drug before an anesthesia is administered—and then, to provide a further drug when the patient comes out of the surgery (i.e., “Drug Rescue Therapy”). Unfortunately, even this does not always work. As is also understood, similar administrations of drugs are made to prevent chemo induced nausea and vomiting (CINV), but with comparable less-than-optimum results.

Recent research in the field report a degree of success in the use of acupressure to alleviate postoperative nausea and vomiting, especially in ambulatory surgery. Likewise, research has indicated a degree of success of acupressure in preventing nausea and vomiting in chemotherapy treatments, beyond the use of antiemetic drugs. In each instance the use of acupressure reduced the incidence of nausea and vomiting.

Although acupressure wrist bands have been utilized in the past for the casual prevention of nausea (as for travel motion sickness and early pregnancy nausea prevention), they typically sell for about $10.00-$12.00 per pair, one for each hand. As will become clear from the following description, however, the acupressure relief afforded by the present invention not only obviates the hassle accompanying a medical patient's attempted use of such bands, but also provides a concomitant reduction in cost when tailored for use by a medical or surgical staff. As will become clear, the acupressure relief of the invention is provided—not by a band which encircles the wrist but through a strip which lies along the inside of the wrist—yet at the same time being disposable, hypoallergenic and skin contact friendly. As will also become clear, the strip of the invention is dimensionable for use both by people, and additionally by dogs and cats who frequently exhibit the same symptoms of motion sickness and of nausea and vomiting after surgery.

### SUMMARY OF THE INVENTION

Intended for positioning along the wrist, at the pericardium 6 (P6 meridian point on the wrist) or at the Pe6 meridian point on the foreleg of the dog or cat, the disposable acupressure relief strip of the invention includes a plastic tape substrate of prescribed length and width with an overlying liner of paper. An adhesive lamination (of acrylate composition, for example) is provided as a top surface of the tape substrate and a cutout is provided through the liner to the substrate across the width of the strip. To apply the acupressure, a bumper or contact button is adhered to the lamination, with a raised portion extending up through the cutout. In a preferred construction, the raised contact portion is of a circular configuration, and the papered liner is peelable away from the adhesive lamination at opposing sides of the cutout.

In this preferred construction, the strip includes a plastic tape in the nature of a transparent perforated polyolefin film which is hypoallergenic, which is easy to tear bi-directionally, and which offers strong adhesion and visualization of the skin underneath. The bumper or contact button (which is affixed to the plastic tape) is of polyurethane, having a pressure sensitive adhesive backing and an elastomer coated raised portion which does not irritate the skin where contact is made. The pressure sensitive adhesive backing is of a high performance adhesive of a thin polyester film. With the tape further being perforated, the tape can be easily torn into a 5 inch length and 1 inch width defining a preferred construction of the invention in providing the acupressure relief.

As will be appreciated by those knowledgeable in the art, a pair of such tape-button, strip combinations are utilized together—one along each wrist of a human, for example, or one on each foreleg above the carpal or wrist bone of the dog or cat. An acupressure relief package including two of these strips can thus be provided within a pouch, according to the invention, with instructions for use printed directly on the pouch. One advantage of this will be seen to be that several such pouches could be removed from a box in which they come, to be taken to different operating rooms from a single supply center—without having to worry about the instructions for use only being printed on the box; this way, each member of the professional staff who emplaces or removes the strip on or from a patient has his/her own set of instructions available to them.

As will become clear from the following description, a single embodiment of the invention is described for use on a human, or for use on a dog or cat. As will be seen, the difference lies in the application of the acupressure relief strip, but having the same location of the cutout through which the contact button extends.

### BRIEF DESCRIPTION OF THE DRAWINGS

These and other features of the present invention will be more clearly understood from a consideration of the following description, taken in connection with the accompanying drawings, in which:

FIGS. 1 and 2 are helpful in an understanding of the use of the disposable acupressure relief strip of the invention;

FIGS. 3a and 3b are top and side views of the paper liner and bottom substrate of the plastic tape; and

FIG. 4 is an illustration of the raised bumper contact button which extends through the plastic tape cutout according to the invention.

### DETAILED DESCRIPTION OF THE INVENTION

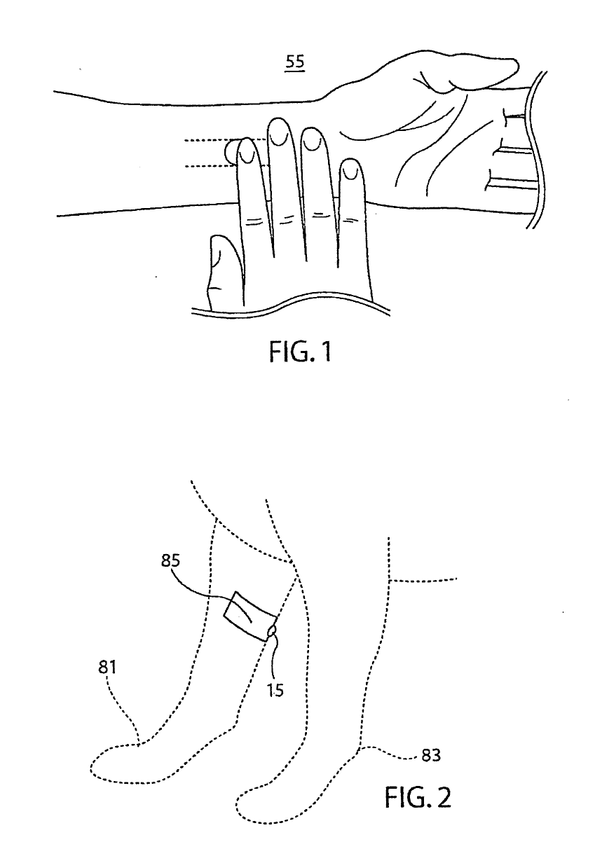

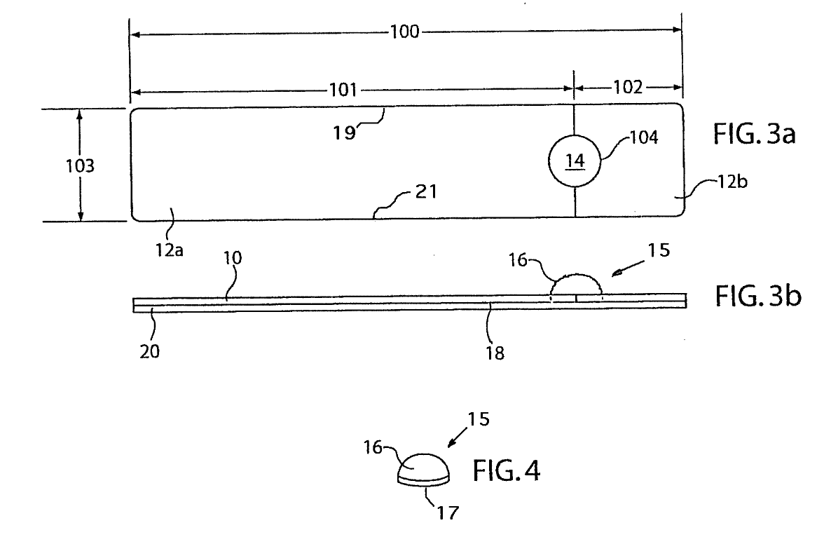

FIGS. 1, 3a, 3b and 4 show the plastic tape substrate of the disposable acupressure relief strip at 20, its overlying adhesive lamination at 18, the paper liner at 10 and the liner cutout at 14. The bumper or contact button is shown at 15, with its raised portion shown at 16.

The positioning of the acupressure relief strip of the invention for use on both wrists of a human follows the placing of the first three fingers on the wrist crease on its inside, to locate where the P6 pressure point is situated between the two flex tendons 55 (i.e., under the index finger). The paper liner 10 of the relief strip is then peeled off on one side 12a of the cutout 14, then off the other side 12b. The raised elastomer coated portion 16 of the contact button 15 that extends through the cutout is then placed face down with its raised contact portion on the P6 pressure point of each wrist. The adhesive lamination 18 that is exposed by the peeling off of the liner 10 is pressed horizontally across the forearm area, along the inside of the wrist, to maintain comfortable pressure on the P6 point throughout. The cutout 14 may be centrally placed along the strip and from top-to-bottom, but offset from side-to-side. The bottom of the contact button 15 is then pressed, so that its raised portion 16 stimulates the P6 point. Applying firm pressure for 30 seconds is usually sufficient, and within minutes the stimulation effect will occur. The process is then preferably repeated on the opposite arm. The contact button 15 includes a pressure sensitive adhesive backing 17 to adhere to the adhesive lamination 18.

In use by the professional medical staff in putting on and then taking off the strip after use, a compress pressure is impacted to the contact button 15 on each wrist before the emetic stimulus or chemo medication is initiated. Investigation has shown that the P6 point is typically activated within 5 minutes from the time pressure is compressed on both contact buttons. After the procedure is completed, the relief strip is simply peeled away from the wrist and removed—heightened by the adhesive lamination being selected as hypoallergenic and skin contact friendly.

As will be appreciated by those skilled in the art, in dimensioning the relief strip of the invention, it is not necessary for the strip to wrap around the user's wrist. What is necessary is that the strip be of sufficient length and width, and the contact button be of sufficient diameter (where the cutout 14 is circular), to overlap in being able to provide downward pressure of the raised contact button at the P6 pericardium point. In one construction of the disposable acupressure relief strip for use by a human, the following dimensions were found useful:

| | |
|---|---|
| Length 100 | 5.0 inches |
| Length 101 | 4.0 inches |
| Length 102 | 1.0 inches |
| Width 103 | 1.0 inches |
| Cutout Diameter 104 | 0.5 inches |

In this manner, the cutout 14 will be seen to be offset positioned one fifth of the length in from one end of the adhesive lamination and film substrate, but centrally positioned between its top and bottom edges 19, 21.

A latex-free, hypoallergenic tape identified by catalog number 1527-1 from the 3M Company has worked quite well as the plastic tape of the invention. A hemispherical button from Action Fabricators Inc. of product number 3002 designation has worked quite well as the bumper or contact button. Such contact button, measuring 0.44 inches in diameter and 0.20 inches in height, together with the 3M tape, provided the acupressure relief desired.

Testing has shown that an acupressure nausea protector of this configuration provided very satisfactory results for more than 24 hours, and with or without the use of antisickness drugs. As will be apparent, besides being used to prevent postoperative nausea and vomiting, and chemo induced nausea and vomiting in a medical procedure environment, the acupressure relief strip of the invention could also be used as a preventer of travel motion sickness and of early pregnancy nausea as well.

When packaged with two such constructions in a heat sealed pouch, the pouch can be imprinted with the following directions:
Nausea Relief Instructions For Adult Men and Women
Intended Use: For Men and Women for the relief of nausea symptoms Related to Surgery, travel, chemotherapy and morning sickness.
1. Locate pressure point on each wrist, using the three middle fingers of your other hand and placing them centrally on the inside of the wrist, starting with the first crease between the two central tendons. The point is located under the index finger.
2. Once located, massage or press down on the point area for 30 seconds to activate the relief.
3. Place with the contact button face down over point.
4. Remove liners from adhesive and apply across the wrist.
5. Repeat with other wrist.

With a pouch packaging two such constructions of the invention for use with dogs and cats, the instructions printed on the back of the pouch may be as follows:
Nausea Relief Instructions For Dogs & Cats
Intended Use: Relief of nausea symptoms related to surgery and travel.
1. Locate pressure point on each foreleg, using your index finger and place it centrally on the deepest part of the back of the foreleg area, above the carpal or wrist bone, between the two central tendons.
2. Once located, massage the point area for 30 seconds to activate relief.
3. Place strip with the contact button face down over point.
4. Remove liners from adhesive and wrap comfortably across leg area.
5. Repeat with other foreleg.
   FIG. 2 shows the forelegs 81 of the dog or cat, the wrist bone 83, and the contact button 15 and strip 85 in place.

The same dimensions used for the disposable acupressure relief strip for humans could be used for the relief strip for these pets as well. The configuration of the strip can then suit both applications.

The instructions for human use and for dog and cat use as set out above could be printed on an instruction card for the heat sealed pouch packaging the two disposable acupressure relief units employed for the acupressure nausea protection of the invention.

While there have been described what are considered to be preferred embodiments of the present invention, it will be readily appreciated by those skilled in the art that modifications can be made without departing from the scope of the teachings herein of a hypoallergenic, skin contact friendly disposable strip which allows continuous acupressure stimulation for nausea protection. For at least such reason, therefore, resort should be had to the claims appended hereto for a true understanding of the scope of the invention.

I claim:

1. A disposable acupressure relief strip for the wrist of a user to mitigate the incidence of nausea and vomiting following one of a chemotherapy treatment or a surgical procedure, comprising:
a perforated polyolefin film substrate of prescribed length and width having an adhesive lamination on a top surface thereof;
a paper liner overlying said adhesive lamination;
a cutout through said liner to said adhesive lamination; and
a polyurethane contact button having a pressure sensitive adhesive backing extending downwardly to said adhesive lamination through said cutout, and an elastomer coated raised portion extending upwardly from said adhesive backing through said cutout.

2. The disposable acupressure relief strip of claim 1 wherein said paper liner is peelable away from said adhesive lamination in a first section along a first side of said cutout, and in a second section along a second side of said cutout.

3. The disposable acupressure relief strip of claim 1 wherein said cutout is offset positioned along the length of said adhesive lamination and said film substrate.

4. The disposable acupressure relief strip of claim 3 wherein said cutout is offset positioned one-fifth of the length in from one end of said adhesive lamination and said film substrate.

5. The disposable acupressure relief strip of claim 3 wherein said cutout is centrally positioned between a top edge and a bottom edge of said adhesive lamination and said film substrate.

6. The disposable acupressure relief strip of claim 3 wherein said cutout is offset positioned one fifth of the length in from one end of said adhesive lamination and said film substrate, and centrally positioned between a top edge and a bottom edge of said adhesive lamination and said film substrate.

7. The disposable acupressure relief strip of claim 6 wherein said film substrate is of a 5.0 inch length and 1.0 inch width, and wherein said cutout is circular of a diameter of 0.50 inches.

8. The disposable acupressure relief strip of claim 1 wherein said film substrate is hypoallergenic and tearable bi-directionally.

9. The disposable acupressure relief strip of claim 8 wherein said adhesive lamination is of an acrylate composition.
